(19) United States
(12) Patent Application Publication
(10) Pub. No.: US 2016/0106300 A1
(43) Pub. Date: Apr. 21, 2016

(54) Flexible Endoscopic Exoskeleton Surgical System

(71) Applicant: Willard S. Noyes, Lexington, IL (US)

(72) Inventor: Willard S. Noyes, Lexington, IL (US)

(21) Appl. No.: 14/886,988

(22) Filed: Oct. 19, 2015

Related U.S. Application Data
(60) Provisional application No. 62/083,181, filed on Nov. 22, 2014.
(60) Provisional application No. 62/065,594, filed on Oct. 17, 2014.

(51) Int. Cl. A61B 1/00; A61B 1/06; A61B 1/055; A61B 18/20; A61B 1/005

(52) U.S. Cl. CPC A61B 1/00087; A61B 18/20; A61B 1/00096; A61B 1/005; A61B 1/055; A61B 1/0669

(57) ABSTRACT

A surgical system includes a flexible endoscope including a lens at a distal endoscope end, a hollow shaft including a proximal shaft end and a distal shaft end, and an instrument spanning the length of the shaft. The instrument includes a handle portion adjacent the proximal shaft end and a tool portion adjacent the distal shaft end. When the endoscope is inserted into the shaft, the lens provides, adjacent the proximal shaft end, direct visualization of the change in condition of the tool portion of the instrument adjacent to the distal shaft end.

### CROSS-REFERENCE TO RELATED APPLICATIONS

This application claims the benefit of priority to U.S. Provisional Application No. 62/065,594 filed on Oct. 17, 2014, and to U.S. Provisional Application No. 62/083,181 filed Nov. 22, 2014, the disclosures of which are incorporated herein by reference.

### BACKGROUND OF THE INVENTION

The present subject matter relates generally to a flexible endoscopic exo-assist surgical system. More specifically, the present application discloses a surgical system including a flexible endoscope removably housed within a shaft including an instrument thereon for viewing the active distal end of the instrument while within a cavity of a patient.

Endoscopes are illuminated devices used to look inside a body cavity or organ. Endoscopes are used in various medical procedures, often in combination with other instruments to enable the medical professional to visualize the performance of the other instruments within the body cavity or organ. For example, nasal endoscopes are used for visualization in procedures involving the sinuses and throat.

The current state of technology directed to nasal endoscopes is to use rigid nasal endoscopes with varying angles of visualization; a medical professional picks the most appropriate fixed angle for each procedure. Another common office endoscopic approach involves insertion of a flexible endoscope with a manually adjustable tip that allows the physician to better control and position the flexible endoscope.

As noted above, the physician may be using the endoscope in conjunction with a tool. For example, the endoscope may be used to allow the physician to view tissue or other matter within a cavity in a patient while using suction or grasping forceps to remove tissue from the cavity.

With respect to rigid endoscopes, the existing technology does not allow for direct visualization and removal of hard to reach sinus tissue or cauterization of hard to reach bleeding sites. Current rigid endoscopes cannot be inserted directly into the frontal sinuses or gain proper visualization of the medial inferior maxillary sinus cavity. Removing laryngeal tissue and foreign bodies as well as tissue in patients with nasopharyngeal stenosis is also difficult because of inadequate visualization and difficulty in reaching desired anatomical sites. Further, rigid endoscopes often distort the surgeon's perspective and are cumbersome to use in conjunction with secondary instruments such as tools in a small cavity.

As a result, ENT physicians are typically unable to directly visualize removal of all maxillary sinus tissue during endoscopic sinus procedures. The surgeon is often handicapped by the rigidity of the endoscope and the angle of visualization when trying to perform tasks in areas difficult to reach with sinus instruments. The surgeon is handicapped even with the available angled rigid scope visualization. In addition, especially in pediatric cases, there is simply not enough room to insert multiple instruments in a nasal passage or sinus opening at the same time.

With respect to flexible nasal endoscopes, current flexible nasal endoscopes are too long and difficult to control in the nose when manipulating simultaneously with other sinus instruments. For example, flexible ear, nose and throat (ENT) endoscopes are too narrow to include a channel for accommodating suction or grabbing forceps. For example, current methods for laryngeal injection of Botox and/or Radeisse hydroxyl-appetite are cumbersome, and require a flexible nasopharyngoscope and a separate needle injector to be manipulated simultaneously in a manner that is cumbersome to the physician and uncomfortable to the patient.

Historically, the typical ENT flexible nasopharyngoscope is used in the office. While the instrument is convenient for visualizing around corners, it does not include channels for accommodating a forceps, laser, or cautery. Any such channel would not be sufficiently wide for the appropriate grasping forceps for necessary tasks. Using a currently available flexible endoscope requires two hands: one hand to manipulate the tip flexion and another hand to stabilize the tip at the nostril. If a physician wants to biopsy, cauterize, or inject under flexible endoscope guidance, the hand at the nostril must be released in order to work the second instrument, balloon, cautery, or suction.

Current technologies fall short because a rigid endoscope cannot be bent and a flexible endoscope cannot be stabilized effectively. Grasping or removing tissue in hard to visualize and hard to reach areas is often done blindly and therefore prone to incomplete tissue removal. The angles are not adjustable and trying to visualize while at the same time manipulating angled instruments is cumbersome and particularly difficult to master for novice surgeons. Accordingly, there is a need for direct visualization of tissue located in areas that are difficulty to reach while simultaneously utilizing additional tools. There is a need for a more precise, accurate, and simplified systems and methods for controlling and manipulating nasal endoscopes for ENT, critical care, and emergency room physicians.

### BRIEF SUMMARY OF THE INVENTION

In order to meet the needs described above, the present subject matter discloses a surgical system and method that provides an endoscope within a shaft including a manipulable instrument thereon for insertion into a cavity of a patient, such as a nasal passage, sinus, or larynx. The surgical systems provides for a single device insertion that includes the endoscope, the shaft for positioning the endoscope, and the instrument for contacting tissue within the cavity so that the physician has direct visualization of a tool portion of the instrument within the cavity through the endoscope.

Using the surgical system of the present application, a physician is able to use one hand to position a distal endoscope end and activate the attached tool portion of the instrument simultaneously. The manipulable instrument is positioned on the shaft so that a tool portion of the instrument extends from a distal shaft end of the shaft. The endoscope is inserted through the shaft until the distal endoscope end exits the distal shaft end and is positioned at or between the distal shaft end and the tool portion of the instrument. The desired angles of the distal endoscope end as well as the tool portion are controlled by changing the angulation of the distal shaft. Further, a lens on the distal endoscope end helps the user to visualize the tool portion of the instrument from the proximal end of the endoscope.

Combining the three elements of the shaft, the endoscope, and the instrument into a single device reduces the amount of space occupied within the cavity, which in some cases may be very limited. Additionally, the system allows for rotating a flexible angled endoscope tip within the shaft, providing a greater degree of visualization and easier access to visualizing all of the surfaces within the cavity. Minimizing the intrusion into the patient is less cumbersome for the physician and more comfortable for the patient.

The surgical system may be disposable or reusable, as will be appreciated by those skilled in the art.

In one embodiment, a surgical system includes a flexible endoscope including a lens at a distal end, a hollow shaft including a proximal shaft end and a distal shaft end, and an instrument spanning the length of the shaft. The instrument includes a handle portion adjacent the proximal shaft end and a tool portion adjacent the distal shaft end. When the endoscope is inserted into the shaft, the lens provides, adjacent the proximal shaft end, direct visualization of the change in condition of the tool portion of the instrument adjacent to the distal shaft end.

In some embodiments, the instrument is manipulable and includes an actuating portion at the handle. Activation of the actuating portion causes a change in condition of the tool portion. In other embodiments, the instrument is fixed.

In some embodiments, the shaft of the surgical system is rigid, malleable, and/or flexible. The shaft may be metallic. Further, the distal shaft end may be moveable relative to a main portion of the shaft. The shaft may include an obturator or other adjusting mechanism for moving or adjusting the distal shaft end.

The instrument may include one of a suction device, a forceps, a scissors, a culture swab, a wire basket, a snare, a cautery device, a microdebrider, a laser or laser tip, a balloon, a drill, a curette, a probe, and a needle. In some embodiments, the instrument includes a locking mechanism that clamps onto the endoscope.

A proximal endoscope end of the endoscope may include an eyepiece and a camera adjacent to the eyepiece that is in visual communication with the lens. The eyepiece may include a connection for a light source that directs light through the endoscope. In some embodiments, the endoscope comprises a fiberoptic endoscope. The shaft may include an irrigation port for clearing the lens of blood and/or debris.

In a further embodiment, a method of performing an endoscopy on a patient includes the steps of: providing a shaft including a proximal shaft end and a distal shaft end, wherein a handle portion and a tool portion of an instrument are at the proximal shaft end and the distal shaft end, respectively; providing an endoscope including an eyepiece at an endoscope proximal end and a lens at an endoscope distal end, wherein the eyepiece is in visual communication with the lens; and inserting the distal shaft end into the patient. The method further includes the steps of inserting the endoscope distal end through the shaft so that it exits the distal shaft end and viewing the tool portion of the instrument through the eyepiece of the endoscope.

In some embodiments, the step of inserting the distal shaft end into the patient precedes the step of inserting the endoscope distal end through the shaft.

In other embodiments, the step of inserting the distal shaft end into the patient comprises inserting the distal shaft end into a nasal passage, a pharynx, a larynx, or a trachea of the patient.

One advantage of the present system is providing direct visualization of a tool portion of an instrument as well as greater angles of visualization in a cavity that is difficult to reach.

Another advantage of the present system is minimizing the space occupied by the endoscope and instrument within the cavity, thereby allowing for easier visualization of the cavity.

A further advantage of the present system is the convenience of using one hand to position both the endoscope and the instrument within the cavity.

A further advantage of the present system is providing a flexible endoscope with a tip that is not manipulable or adjustable, thereby providing the ability to use a thinner, shorter, sturdier, and less expensive endoscope.

This invention will also allow for a more practical, thorough, time efficient manner to visualize and perform standard endoscopic surgical tasks in the office or surgical setting.

Additional objects, advantages and novel features of the examples will be set forth in part in the description which follows, and in part will become apparent to those skilled in the art upon examination of the following description and the accompanying drawings or may be learned by production or operation of the examples. The objects and advantages of the concepts may be realized and attained by means of the methodologies, instrumentalities and combinations particularly pointed out in the appended claims.

### BRIEF DESCRIPTION OF THE DRAWINGS

The drawing figures depict one or more implementations in accord with the present concepts, by way of example only, not by way of limitations. In the figures, like reference numerals refer to the same or similar elements.

FIG. 1 is a side view of an endoscope as known in the art.

FIG. 2 is a perspective view of an embodiment of a surgical system utilizing the endoscope of FIG. 1.

FIG. 3 is a schematic of the surgical system of FIG. 2 inserted into a nasal passage of a patient.

FIG. 4 is a perspective view of a further embodiment of a surgical system.

FIG. 5 is a perspective view of a further embodiment of a surgical system.

FIG. 6 is a schematic of the surgical system of FIG. 5 inserted into a larynx of a patient.

### DETAILED DESCRIPTION OF THE INVENTION

The present disclosure provides a surgical system 100 designed to visualize proper placement of an instrument 128 such as sinus and laryngeal forceps, a cautery device, a suction device, injection needles, etc. More specifically, the surgical systems 100 of the present application provide a instrument 128 including a tool portion 132 that is inserted into a cavity of a patient, such as a nasal passage, sinus, or larynx, and is easily visualized using an endoscope 102. While the illustrated embodiments include instruments used primarily in otolaryngology, other embodiments may include instruments used in other surgical specialties such as, for example, urology, gastroenterology, orthopedics, and general surgery.

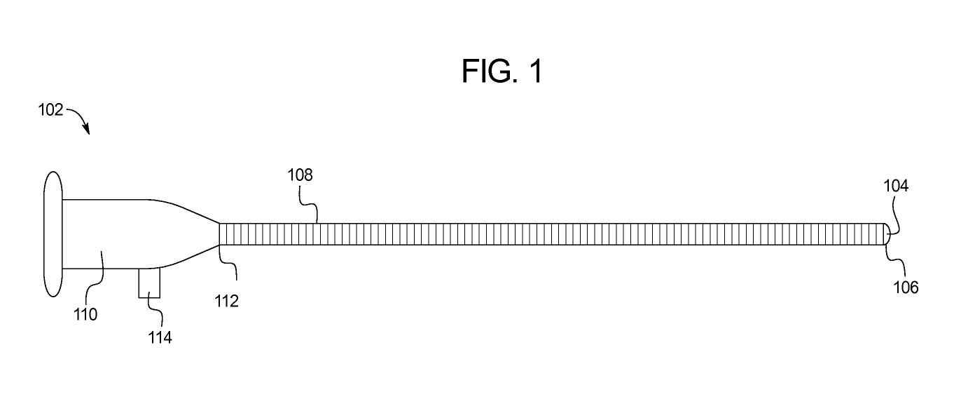

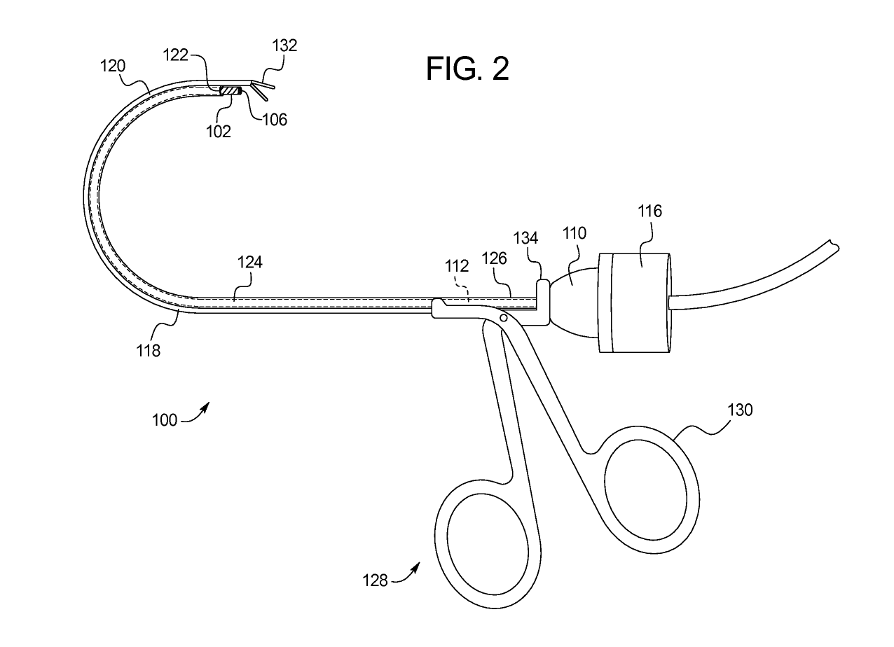

FIG. 1 illustrates a flexible endoscope 102 including a lens 104 disposed at a distal endoscope end 106. In the illustrated embodiment, the endoscope is flexible throughout its length and does not include a lever or any other controlling mechanism that allows for manipulation of the distal endoscope end 106. The length of the endoscope 102 is about 6 inches to about 10 inches, preferably about 8 inches. The endoscope 102 may include optical fibers 108, with the image captured by the lens 104 being transmitted through the optical fibers 108 to an eyepiece 110 at a proximal endoscope end 112 of the endoscope 102. The eyepiece 110 may include a connection 114 for a light source that directs light through the optical fibers 108 and is emitted from the distal endoscope end 106. A camera 116 shown in FIG. 2 may be positioned on the eyepiece 110. In other embodiments, the endoscope 102 may include a flexible tube that houses a lens system in lieu of the optical fibers 108. Further, the endoscope 102 may include image guidance technology.

The flexible endoscope 102 may be selected from any number of currently available endoscope lengths with varying calipers to accommodate adult and pediatric patients, as well as, nasal, tracheal, trachea-stomal, and laryngo-pharyngeal applications. The distal endoscope end 106 may be attached to a device (not shown) that enables the user to adjust the positioning of the distal endoscope end as well as provides a medication-dispensing source, an air source for balloon inflation, or the like. The distal endoscope end 106 may be encased in a catheter or sheath as disclosed herein or any conventional catheter or sheath.

Referring to FIG. 2, the surgical system 100 includes a hollow shaft 118 that acts as a chamber to accommodate the endoscope 102 in an exoskeleton-type fashion. Different embodiments of the system would allow for different shaft angles and tool functions. The shaft 118 may be rigid, malleable, flexible, or any combination thereof. In one embodiment, an adjustable portion 120 of the shaft 118 near a distal shaft end 122 may be moveable relative to a main portion 124 of the shaft 118. Adjustment of the adjustable portion 120 to various angles prior or subsequent to insertion may allow for easier insertion and/or manipulation, particularly in smaller openings. The shaft 118 may include an inner obturator for manual adjustment or an automatic adjustment mechanism built into the shaft at a proximal shaft end 126.

During use, the endoscope 102 extends through the shaft 118 such that the distal endoscope end 106 extends from the distal shaft end 122 and the proximal endoscope end 112 extends from the proximal shaft end 126. The distal endoscope end 106 may include a flexible, angled end that straightens during insertion through the shaft 118 and returns to the angled position once it passes the distal shaft end 122. The angled end of the distal endoscope end 106 allows for even greater angles of visualization, particularly along the floor of the frontal and maxillary sinuses. The flexible endoscope 102 may be rotated within the shaft 118 to allow for 360 degrees of visualization.

An instrument 128 spans the length of the shaft 118 and includes a handle portion 130 and a tool portion 132. In some embodiments, instrument 128 is manipulable and the handle portion 130 comprises an actuating portion. The actuating portion 130 and a tool portion 132 are adjacent to the proximal shaft end 126 and the distal shaft end 122, respectively, of the shaft 118. Activation of the actuating portion 130 causes a change in condition of the tool portion 132. The manipulable instrument 128 may include one of a suction device, grasping forceps, cutting forceps, Kerrison forceps, curettes, a culture swab, a wire basket, a snare, a cautery device, a needle, a laser, balloon dilators, powered instrumentation tips, drills, or any suitable tool. In other embodiments, the instrument 128 is fixed such that the tool portion 132 of the instrument 128 is not manipulable. For example, the tool portion may include a curette, a probe, or a laser tip. With either a manipulable or a fixed instrument, action at the handle portion may influence an outcome or movement at the tool portion of the instrument. In any embodiment, the tool portion of the instrument may be angled in any direction, such as to the right, left, up, or down of the distal shaft end, and to any degree between 0 and 90°, such as 10°, 30°, or 60°. The instrument 128 may also include a locking device 134 that clamps the instrument 128 onto the endoscope 102.

During use, the tool portion 132 of the instrument 128 is directly in front of the distal shaft end 122 such that the image captured by the lens 104 of the endoscope 102 includes the tool portion 132 of the instrument 128. In the embodiment illustrated in FIG. 2, the instrument 128 is a forceps, and the actuating portion 130 and the tool portion 132 are handles and blades, respectively.

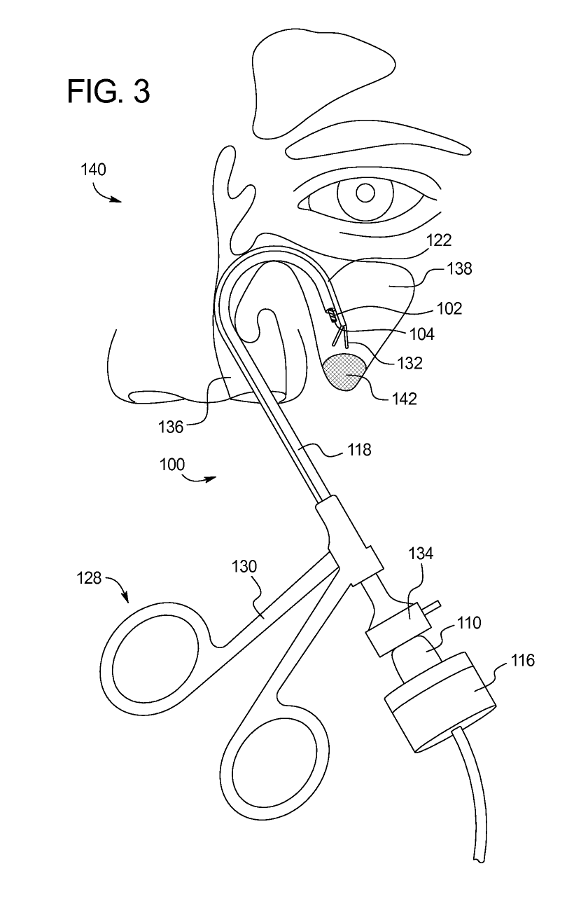

As shown in FIG. 3, the shaft 118 of the surgical system 100 may be inserted through a nasal passage 136 and into a sinus cavity 138 of a patient 140. The blades 132 of the forceps 128 are positioned near the tissue 142 within the maxillary sinus 138 that needs to be removed. The positioning of the blades 132 of the forceps 128 directly in front of the lens 104 of the endoscope 102 allows the physician to visualize the blades 132 as they contact the tissue 142. The physician can then activate the handles 130 of the forceps 128 while viewing the blades 132 as they grasp or cut the tissue 142.

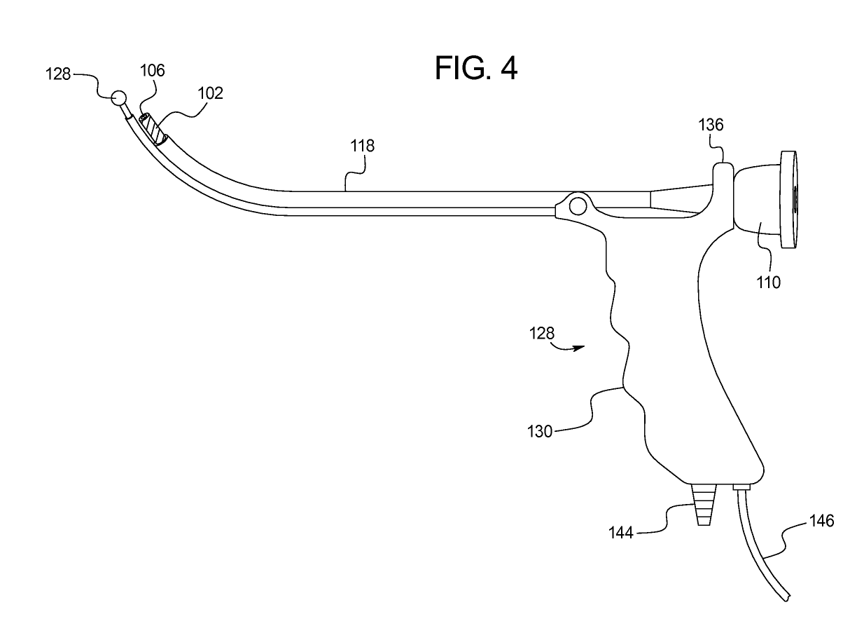

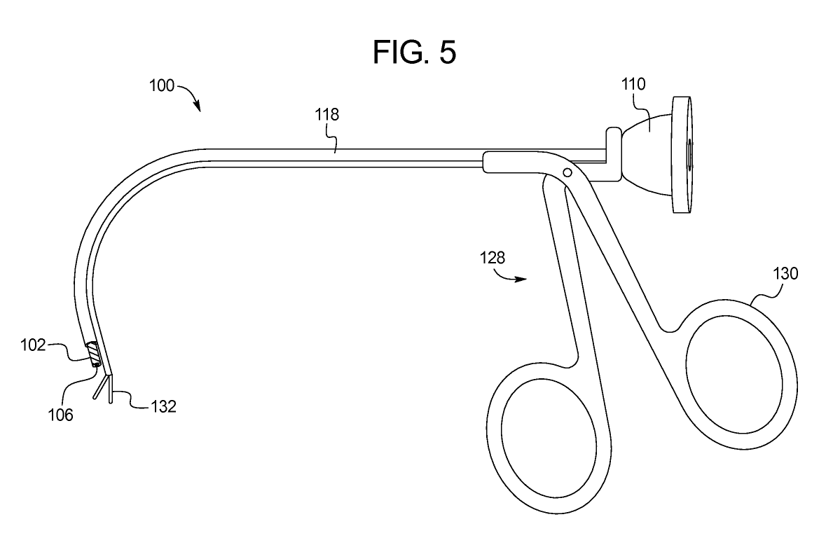

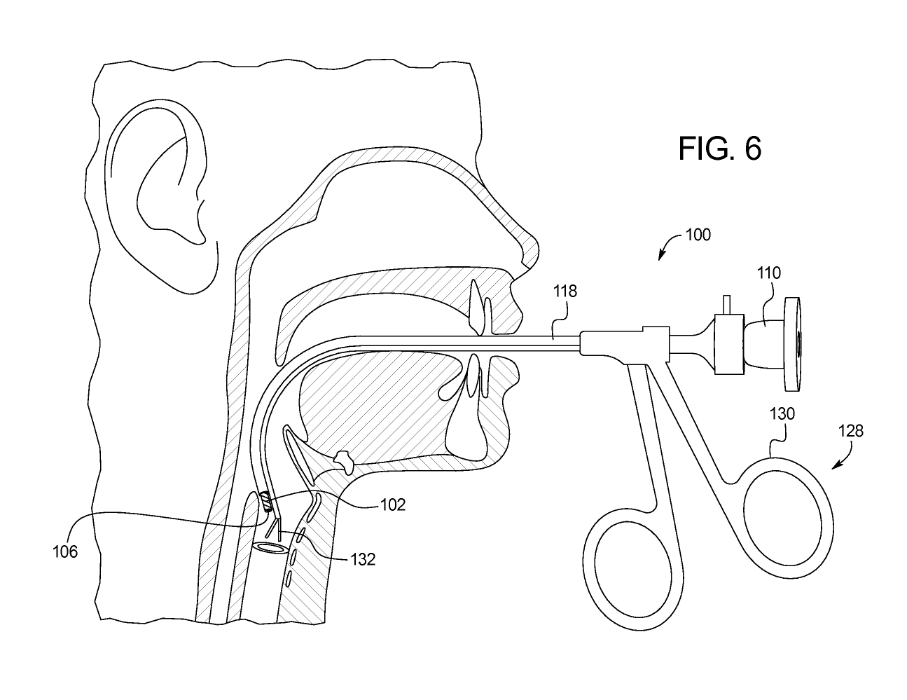

In the surgical system illustrated in FIG. 4, the manipulable instrument 128 is a suction cautery device. The actuating portion 130 is a handle with a suction port 144 and a cautery cord 146. The tool portion 132 is an electrode. In this embodiment, the locking mechanism 136 of the instrument 128 clasps the eyepiece 110 of the endoscope 102. FIGS. 5 and 6 illustrates an embodiment of the surgical system including a laryngeal forceps 128. The surgical system may also include an injection needle.

It should be noted that various changes and modifications to the presently preferred embodiments described herein will be apparent to those skilled in the art. Such changes and modifications may be made without departing from the spirit and scope of the present invention and without diminishing its attendant advantages.

I claim:

1. A surgical system comprising:
a flexible endoscope including a lens at a distal endoscope end;
a hollow shaft including a proximal shaft end and a distal shaft end; and
an instrument spanning the length of the shaft including a handle portion adjacent the proximal shaft end and a tool portion adjacent the distal shaft end;
wherein, when the endoscope is inserted into the shaft, the lens provides, adjacent the proximal shaft end, direct visualization of the tool portion of the instrument adjacent to the distal shaft end.

2. The surgical system of claim 1, wherein the instrument is manipulable and includes an actuating portion at the handle, wherein activation of the actuating portion causes a change in condition of the tool portion.

3. The surgical system of claim 1, wherein the instrument is fixed.

4. The surgical system of claim 1, wherein the shaft is rigid.

5. The surgical system of claim 4, wherein the shaft is metallic.

6. The surgical system of claim 1, wherein the shaft is malleable.

7. The surgical system of claim 1, wherein the shaft is flexible.

8. The surgical system of claim 1, wherein the distal shaft end is moveable relative to a main portion of the shaft.

9. The surgical system of claim 8, wherein the shaft includes an obturator for moving the distal end.

10. The surgical system of claim 8, wherein the shaft includes an adjusting mechanism for moving the distal end.

11. The surgical system of claim 1, wherein the instrument includes one of a suction device, a forceps, a scissors, a culture swab, a laser, a wire basket, a snare, a cautery device, a microdebrider, a needle, a drill, a curette, a probe, and a laser tip.

12. The surgical system of claim 1, wherein the instrument includes a locking mechanism that clamps onto the endoscope.

13. The surgical system of claim 1, further comprising an eyepiece at a proximal endoscope end.

14. The surgical system of claim 13, further comprising a camera adjacent to the eyepiece that is in visual communication with the lens.

15. The surgical system of claim 14, wherein the endoscope comprises a fiberoptic endoscope.

16. The surgical system of claim 15, wherein the eyepiece includes a connection for a light source that directs light through the endoscope.

17. The surgical system of claim 1, wherein the shaft includes an irrigation port for clearing the lens of blood and/or debris.

18. A method of performing an endoscopy on a patient comprising:
providing shaft including a proximal shaft end and a distal shaft end, wherein a handle portion and a tool portion of an instrument are at the proximal shaft end and the distal shaft end, respectively;
providing an endoscope including an eyepiece at an endoscope proximal end and a lens at an endoscope distal end, wherein the eyepiece is in visual communication with the lens;
inserting the distal shaft end into the patient;
inserting the endoscope distal end through the shaft so that it exits the distal shaft end;
viewing the tool portion of the instrument through the eyepiece of the endoscope.

19. The method of claim 18, wherein the step of inserting the distal shaft end precedes the step of inserting the endoscope distal end through the shaft.

20. The method of claim 18, wherein the step of inserting the distal shaft end comprises inserting the distal shaft end into one of a nasal passage, a pharynx, a larynx, and a trachea of the patient.
